(19) United States
(12) Patent Application Publication
(10) Pub. No.: US 2008/0081798 A1
(43) Pub. Date: Apr. 3, 2008

(54) Pharmaceutical combinations for the treatment of head-aches and migraine attacks as well as blisters and packs that contain them

(76) Inventor: Abouch Valenty Krymchantowski, Rio de Janeiro (BR)

Correspondence Address: GOTTLIEB RACKMAN & REISMAN PC, 270 MADISON AVENUE, 8TH FLOOR, NEW YORK, NY, 10016-0601, US

(21) Appl. No.: 11/542,656

(22) Filed: Oct. 3, 2006

(51) Int. Cl. A61K 31/60; A61K 31/405; A61K 31/4152; A61K 31/4172

(52) U.S. Cl. 514/165; 514/381; 514/400; 514/568; 514/569; 514/420; 514/406; 514/554

(57) ABSTRACT

A pharmaceutical combination comprising at least a triptan and the trimebutine compound [hydrogenated maleate of 2-dimethyl-amino-2-phenylbutyl-3,4,5-trimetoxybenzoate] and an anti-inflammatory or simple analgesic for the treatment of attacks of migraine and other headaches is provided.

More particularly, the present invention discloses a pharmaceutical combination comprising triptan and trimebutine for the treatment of migraine attacks.

The present invention further discloses blisters and packs containing said pharmaceutical combinations.

The present invention refers to a pharmaceutical combination comprising at least a triptan and the trimebutine compound [hydrogenated maleate of 2-dimethyl-amino-2-phenylbutyl-3,4,5-trimetoxybenzoate] and an anti-inflammatory or simple analgesic for the treatment of attacks of migraine and other headaches.

More particularly, the present invention discloses a pharmaceutical combination comprising triptan and trimebutine for the treatment of migraine attacks.

The present invention further discloses blisters and packs containing said pharmaceutical combinations.

### DESCRIPTION OF THE PRIOR ART

The headaches are defined as localized or diffuse pain, in several parts of the head, neck and face and are not limited to the area of distribution of any nerve. More than 90% of people exhibit at least 1 episode of headache per year and this is why they are the most common reason for seeking neurological help in the world and, in some centers, the second largest complaint in any medical specialization.

The impact caused by headaches, especially migraine, on people's social and economic life is huge. Only in the United States, it is estimated that the direct costs associated with consultations, medicines and physicians are around $10 billion a year. When added to the indirect costs caused by absenteeism and the loss of productivity, such figures increase to $27 billion a year. European studies show that people with migraine lose on average up to four days of work per year as a result of the crises of pain not counting the days they work with pain therefore, reducing their productivity. Those who suffer from chronic headache also have incalculable social damages. Anxiety anticipated by the possibility of having a crisis of pain leads to the habit of avoiding not only professional commitments but also leisure activities, family reunions and trips.

There are more than 150 different types of headaches, the most common of which are the primary headaches. The primary headaches are those caused by biochemical abnormalities of the brain itself leading to pain due to the malfunctioning of the neurotransmitters and/or their receptors. Migraine, cluster headache, tension-type headaches, paroxysmal hemicrania, among others, are some types of primary headache.

Migraine is a genetically transmitted and inherited brain disease. Although in only one more rare type of migraine, called familial hemiplegic migraine, chromosome 19 and gene (CACNA1A) that encodes the formation of the ionic calcium channels have been identified as responsible for their transmission from one patient to the other, it is currently accepted that the other types of migraine, including the more common ones, such as migraine without aura, are also inherited by genes. Recently, chromosome 1, in the gene that encodes the ATPase sodium pump, was revealed to be changed in European studies.

The migraine attacks are usually presented as pulsatile or throbbing pain (pressure-like or constriction-like) in the frontal and temporal regions, being present in more than one side of the head (in 40% of the patients, it is on both sides) and with moderate to severe or severe intensity.

It usually debilitates the patient for normal activities, and it aggravates with effort or physical activities, starting mild and progressive: the migraine attacks last on average from 4 to 72 hours, when not treated or ineffectively treated, and in general subside gradually. They are associated with at least two of the following symptoms: nausea or vomiting, intolerance to light or noise (photo- and phonophobia).

The migraine attacks may present 4 different phases: (1) prodrome phase with prodomal symptoms, (2) aura phase with transient neurological signs and symptoms, (3) headache phase with associated characteristics and (4) recovery or postdrome phase frequently associated with rest or sleep.

Cluster headache is the most intense type of headache described by medicine; usually patients describe it as a “piercing and penetrating stab in the eye.” Pain is presented in rhythmic form making some patients know when to expect the next attack, causing great anticipatory anxiety. It is not as common as other primary headaches, such as migraines and tension-type headaches. The causes of this pain are still unknown, but there seems to be a dysfunction in a nucleus of an important brain structure called hypothalamus.

There are recent studies suggesting the relationship between cluster headaches and sleep disorders, such as apnea and snoring. The mechanisms that trigger the attacks are unknown, but during the attacks there are edema-type changes (swelling) and e tortuosity of the internal carotid artery wall in certain parts of its trajectory inside the skull. It is already known that the cluster headache is not caused by allergies, smoking, alcohol and genetic inheritance (despite the recent works suggesting the genetic connection between cluster and migraine patients). It is common for these patients to be wrongly diagnosed as sufferers of migraine, trigeminus neuralgia, atypical facial pain and dental occlusion problems, often being submitted to wrong therapies, such as laser, anti-epileptic medication, surgeries, dental treatments, among others. It affects more men than women at a 9-10 to 1 rate. This pain normally starts between 20 to 40 years of age.

The tension-type headaches are caused by unbalances in the brain's chemical functioning and have no direct relationship with emotional tension, but can be related to exaggerated, abnormal and sustained tension or contraction of muscle groups of the shoulders, neck, scalp and even face. It is the most common type of headache, but because it is not so intense and disabling, its sufferers, even the chronic ones, do not seek medical help as often as the migraine sufferers and frequently make excessive use of analgesics and other drugs on their own; if the latter were more effective and provided faster and more consistent relief, they would probably be less used.

There are studies suggesting that these pains, when chronic, result from biochemical abnormalities of brain areas involving the analgesic system of the brain itself (antinociceptive system). This system produces endorphin and is innervated by neurons that use serotonin as a chemical neurotransmitter. The deficiency of serotonin would lead to the hypofunction of this analgesic system and the patient would present this headache (this mechanism is also found in patients with migraine).

There are basically two kinds of tension-type headache: episodic and chronic. The episodic tension-type headaches are extremely common, moderate and generally are not disabling. However, they become problematic and uncomfortable when they start occurring more than 15 days a month, configuring the chronic character. The episodic tension-type headaches affect 87% of the general population, according to a study developed in Denmark. There are epidemiological studies stating the prevalence of these headaches in 84% of women and 67% of men. The chronic tension-type headache, in turn, is much less common, affecting 3% of the adult population, but generating much more disability and suffering than the acute type. There are studies that prove that the excessive use of moderately effective analgesics leads to the development of chronic headache in the sufferers of the episodic forms.

The table below shows a comparison between the three major types of primary headache.

TABLE 1
Comparison between the three types of primary headache

| | Tension-type headache | Migraine | Cluster headache |
|---|---|---|---|
| Prevalence in the | 65–87% | 12–15% | 0.1–0.4% |
| population | | | |
| Most common age | 20–50 | 20–40 | 20–40 |
| range | | | |
| Gender | more women | more women | more men |
| Family history | frequent | frequent | not frequent |
| Frequency | variable | variable | from 1 to 5 times/day |
| Triggering factors | stress or tiredness | stress, menstruation, certain types of food, alcohol, | alcohol, only during clusters |
| | | climate changes, light, odors etc. | |
| Location | bilateral, frontal, neck | normally unilateral, sometimes bilateral | unilateral, orbital, supraorbital, |
| | and top of the head | | temporal and malar |
| Intensity | mild to moderate | moderate to intense | very intense |
| Aggravating factors | stress, excess of | movements, sway and lower the head and physical | lie down |
| | work and muscular | effort | |
| | effort | | |
| | Tension-type headache | Migraine | Cluster headache |
| Associated | nausea or photophobia | nausea, sometimes vomiting, photophobia, phonophobia | red eyes, tearing, nasal congestion |
| symptoms | or phonophobia | and/or phobia to odors etc. | and running nose, sweating |
| | may occur. Only 1 of | | of the face, constricted pupil, |
| | them may be present | | drooping eyelids on the same |
| | | | side of the pain, facial edema |
| Duration of the | from a few hours to | from 4 to 72 hours | from 15 to 180 minutes |
| pain | days | | |

The secondary headaches, on the other hand, caused by problems in any region of the body, can have several causes. Brain tumors, meningitis, aneurisms, problems in the eyes, ears, throat and even a simple cold may be responsible for secondary headaches.

Fortunately, almost 90% of the existing headaches belong to the group of primary and functional (non-organic) headaches, such as, for instance, migraine, tension-type headache and cluster headache. The other 10% are among the several types of headache, including some caused by severe diseases of the head itself or other parts of the body.

Currently, there are many medicaments to treat headaches. For example, non-specific treatments including aspirin, paracetamol, non-steroidal anti-inflammatory drugs, opioids and analgesic combinations with caffeine and even barbiturates for the treatment of migraine and the specific ones, including ergotamines, dihydroergotamines and triptans for the treatment of primary headaches, such as migraine and cluster headaches. These latter, however, do not treat the other types of headaches.

U.S. Pat. No. 6,586,458 describes methods for treating headache sufferers comprising the administration of compositions containing a 5-HT agonist (triptan) and a long-acting, non-steroidal anti-inflammatory drug.

The patent application US 2004180089 is directed to pharmaceutical tablets in multiple layers for treating pain and particularly for treating pain associated with migraine, in which a non-steroidal, anti-inflammatory drug and a triptan are present in different and separated layers. The layers are in a side-by-side configuration, which allows the dissolution of triptan and the anti-inflammatory drug in an independent and immediate way.

U.S. Pat. No. 6,077,539 comprises a non-vasoactive, supravasoactive syndrome minimized dosage form for treatment of migraine in a human comprising (i) rapid availability metoclopramide in at least about an effective local gastrointestinal amount; (ii) at least one long acting non-steroidal anti-inflammatory drug such as naproxen sodium in a therapeutically effective amount; (iii) wherein said dosage form is a coordinated dosage form; and, (iv) wherein the dosage form is free from 5HT agonist vasoactive agents, and preparation thereof. However, metoclopramide is an anti-nausea drug with brain penetration that frequently causes asthenia and its excessive use may cause the so-called extrapyramidal syndrome.

However, the objective of the present invention is to provide a totally new pharmaceutical combination comprising at least a triptan and trimebutine, in addition to the anti-inflammatory or simple analgesic to treat attacks of primary headache and migraine.

Although the combination of triptan and non-steroidal anti-inflammatory drugs is known for the specific treatment of headaches, the association of the trimebutine compound with these two compounds accounts for the differential. This compound, until now used for the treatment of abdominal pain, including the irritable colon syndrome, is incorporated into the pharmaceutical combination of the present invention resulting in a greater speed of action and efficacy in the treatment of headache attacks.

This combination of the three compounds represents a milestone in the oral treatment of attacks of headache and migraine, and also greatly improves the existing superiority of the combination of a triptan and an anti-inflammatory or simple analgesic compared to the isolated use of each one of these two pharmacological agents.

### BRIEF DESCRIPTION OF THE INVENTION

One objective of the present invention is to provide a pharmaceutical combination comprising at least a triptan and trimebutine, and an anti-inflammatory or simple analgesic for the treatment of attacks of headache and migraine.

Another more specific objective of the invention is to provide a pharmaceutical combination comprising a triptan and trimebutine for the specific treatment of migraine attacks.

Still another objective of the present invention is to provide blisters and packs containing said pharmaceutical combinations.

### BRIEF DESCRIPTION OF THE DRAWINGS

FIG. 1 discloses the percentage of patients (among 32 patients suffering from migraine) who were without pain after 1 and 2 hours. One part of these patients was treated only with rizatriptan and the other part with the combination of rizatriptan and trimebutine.

FIG. 2 discloses the percentage of all the attacks (among the 67 migraine attacks) treated with the combination of rizatriptan and trimebutine, without pain in 1 and 2 hours. And the percentage of patients treated only with rizatriptan, without pain in 1 and 2 hours.

FIG. 3A discloses the percentage of patients (among the 32 patients suffering from migraine) who were free from nauseas caused by migraine attacks after 1 hour. One part of these patients was treated only with rizatriptan and the other part with the combination of rizatriptan and trimebutine.

FIG. 3B discloses the percentage of patients (among the 32 patients suffering from migraine) who were free from photophobia caused by migraine attacks after 1 hour. One part of these patients was treated only with rizatriptan and the other part with the combination of rizatriptan and trimebutine.

### DETAILED DESCRIPTION OF THE INVENTION

In the practice of the invention, a pharmaceutical combination comprising an effective amount of a triptan, an effective amount of trimebutine and an effective amount of an anti-inflammatory or simple analgesic, in which said compounds are individually arranged into three different tablets or capsules, which are administered simultaneously and randomly, or arranged only in one capsule or tablet, is used for treating attacks of headache and migraine. The invention further discloses blisters containing one or three tablet(s) or capsule(s) as described above and packs containing said blisters.

This combination offers therapeutic effects and relief of the symptoms in a very superior manner when only a triptan or only an anti-inflammatory or simple analgesic is used. Said combination also promotes efficacy and greater speed of action when compared to the combined use of a triptan and a non-steroidal, anti-inflammatory drug or simple analgesic.

In a preferred embodiment of the invention, a pharmaceutical combination comprising an effective amount of a triptan and an effective amount of trimebutine, in which said compounds are individually arranged into two different tablets or capsules, which are administered simultaneously and randomly, or arranged only in one capsule or tablet, is used for treating migraine attacks. The invention further discloses blisters containing one or two tablet(s) or capsule(s) as described above and packs containing said blisters.

This combination offers therapeutic effects and relief of the migraine symptoms in a very superior manner when compared to the combination of a triptan and a non-steroidal anti-inflammatory drug or analgesic.

The association of these three compounds increases the speed of absorption of triptan and/or the simple anti-inflammatory or analgesic, since trimebutine has no penetration and action in the brain, but leads to an increase in the speed of gastric emptying and the motor regulation of the movements of the esophagus, stomach and even the intestine when compared to the already-known prior-art medicaments. It should be emphasized that, during the migraine attack, there is the gastroparesis phenomenon, in which the stomach dilates and diminishes its emptying movement greatly impairing the absorption of any orally-taken drug, causing nauseas and vomiting, so characteristic of the migraine.

In addition to combating the already cited gastroparesis occurring in the migraine attacks, nauseas and even vomiting resulting from it, the greater speed of action of the present invention can provide the pharmacological effect of the drug(s) before the development of the process called peripheral and central sensitization, which also occurs in 2 to 4 hours of a migraine attack and which, when present, prevents the triptan from functioning effectively or enables it to work, but does not avoid the return of the pain before 24 hours, even if the pain has improved (phenomenon called recurrence). Trimebutine is a specific agonist of the opioid receptors of the plexus of Meissner and Auerbach existing in the mucosa of the entire gastrointestinal tract and does not penetrate the hematoencephalic barrier (in the brain) being safely used in newborns and elderly suffering from diseases in the cited tract.

The triptan can be selected from the group consisting of sumatriptan, naratriptan, zolmitriptan, rizatriptan, eletriptan, almotriptan and frovatriptan, among others. Preferably, sumatriptan in the range of 40 to 60 mg, more preferably in the range of 45 to 55 mg; naratriptan in the range of 3 to 4 mg, more preferably in the range of 3.5 to 4 mg; and rizatriptan in the range of 8 to 12 mg, more preferably in the range of 9 to 11 mg. According to the present invention, the most preferred triptan is sumatriptan.

Triptans are selective agonists of the 5-HT1B/1D serotoninergic receptors that promote the constriction of the blood vessels dilated during the attack of migraine and some other primary headaches.

The anti-inflammatory may be selected from the group consisting of meloxican in the range of 8 to 20 mg and, more preferably, in the range of 10 to 20 mg; ibuprofen in the range of 500 to 1000 mg, more preferably, in the range of 600 and 800 mg; lysine clonixinate in the range of 200 to 300 mg, more preferably, in the range of 250 to 300 mg; indometacin in the range of 50 to 80 mg, more preferably, in the range of 60 to 70 mg; naproxen sodium in the range of 500 to 1000 mg, more preferably, in the range of 500 to 600 mg; rofecoxib in the range of 40 to 60 mg, more preferably, in the range of 45 to 55 mg; celecoxib in the range of 180 to 260 mg, more preferably, 200 to 250 mg and cetoprofen in the range of 50 to 120 mg, more preferably, in the range of 50 to 100 mg. According to the present invention, the most preferred anti-inflammatory is meloxican.

The anti-inflammatory drugs act in the synthesis of the chemical mediators of inflammation and pain, such as prostaglandins and neurokinins, combating the inflammatory process and the facilitation of the transmission of the pain to the brain, which occur during the attacks of migraine and other primary headaches.

The analgesic may be selected from the group consisting of paracetamol in the range of 500 to 1500 mg, more preferably, in the range of 700 to 1000 mg and acetylsalicylic acid and salicylates in the range of 500 to 1500 mg, more preferably, in the range of 700 to 900 mg.

The simple analgesics present a central action, in the thalamus, diminishing the afference of painful information that ascends to the brain during the headache attacks. They also have anti-inflammatory action to a lesser extent.

The trimebutine employed in the present invention is the hydrogenated maleate of 2-dimethyl-amino-2-phenylbutyl-3,4,5-trimetoxybenzoate, and its corresponding stereoisomers may be used, being employed in the range of 150 a 250 mg, more preferably, in the range of 180 to 220 mg.

Trimebutine acts on the opioid receptors of the plexus of Meissner and Auerbach promoting the regulation of the esophageal, gastric and intestinal motility that is impaired during the attack of migraine and other primary headaches. This results in a faster gastric emptying and a faster absorption of the active drugs and reduction of nausea and vomiting.

The types of headache that the combinations of the present invention intend to treat are: attacks of migraine, attacks of acute and chronic tension-type headache, cluster headache attacks, intense exacerbations of hemicrania continua and acute and chronic paroxysmal hemicrania, exacerbations of new daily and persistent headache, secondary headache attacks caused by simple and mild pathologies, such as headache caused by the excessive ingestion of alcohol, flu, common cold, high altitude etc.

The dosage to be used with the single, double or triple formulation is of at most twice a day and at most three times a week. In fact, this must be the dosage of every medicament for headache attacks with the objective of avoiding the excessive use of medication and the development of the chronic daily headache, besides the side effects of any painkiller.

The following example illustrates the invention, but does not intend to limit its scope.

### EXAMPLE 1

### Rizatriptan and Trimebutine for Treating Migraine in a Double-Blind, Randomized, Cross-Over and Placebo-Controlled Study

Thirty-two patients (24 women and 8 men, aged between 21 and 61, average=39.7) suffering from migraine according to the criteria of the International Headache Association were randomized to treat 2 consecutive moderate or severe attacks with a tablet of 10 mg of rizatriptan and a capsule of 200 mg of trimebutine and 2 attacks with the same amount of triptan and placebo, and vice-versa. The severity of the attacks, as well as the presence of nauseas and photophobia at the moment of ingesting the drug after 1, 2 and 4 hours were collected.

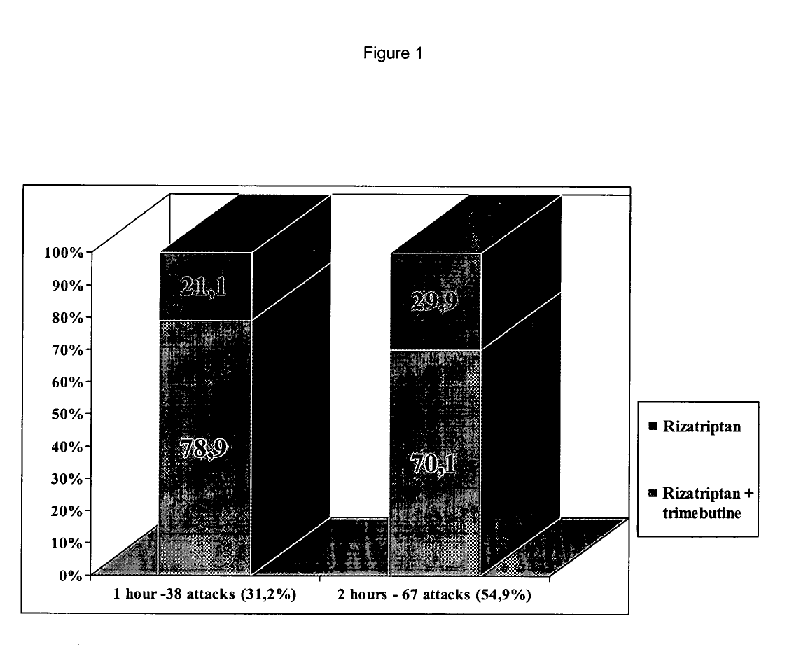

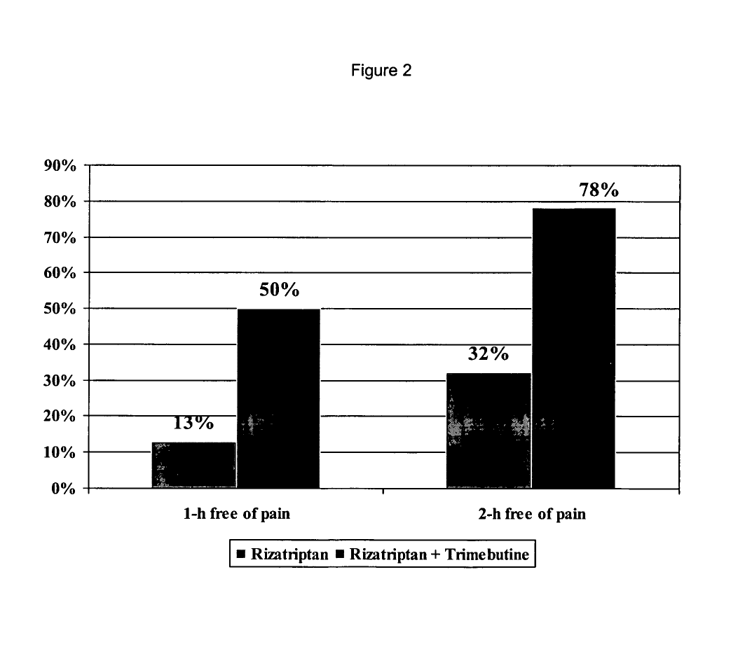

Sixty-four attacks were treated with each drug regimen (total of 122 moderate or severe and 6 mild, the latter, therefore, not being assessed). Of the 67 attacks, 70.1% of the patients that were treated with the combination and 29.9% only with rizatriptan (p<0.0001) were without pain after 2 hours. Of the 38 attacks without pain after 1 hour, 78.9% of the patients were treated with the combination and 21.1% only with rizatriptan (p<0.001)—see FIG. 1.

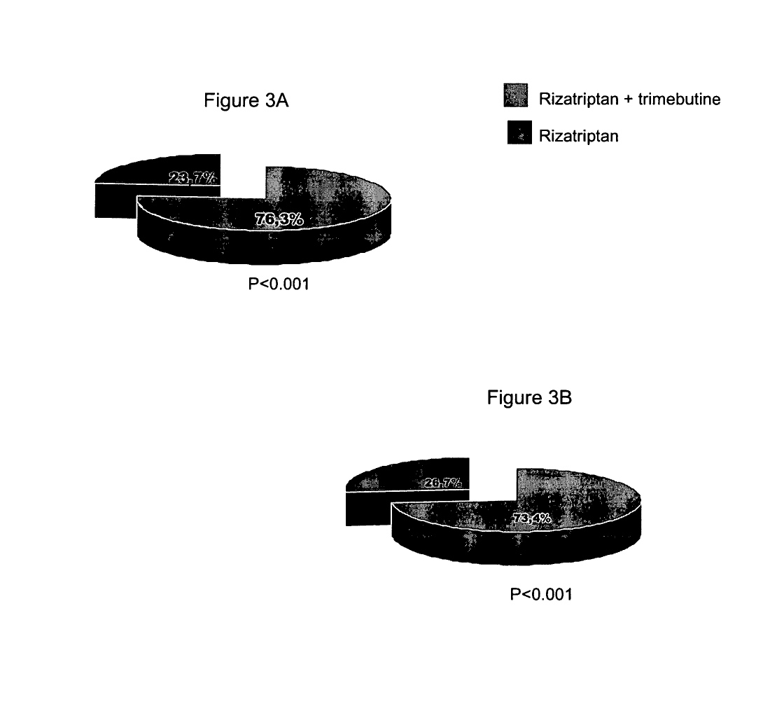

With regard to nausea and photophobia, the combination presented a better efficacy when compared to the treatment only with rizatriptan (76.3% versus 23.7% free from nausea (see FIG. 3A) and 73.4% versus 26.7% free from photophobia (see FIG. 3B) after 1 hour—p<0.001 for both comparisons). The recurrence of the migraine was similar among the two regimens and adverse events were present in 18 attacks treated with rizatriptan and 21 attacks treated with the combination. Vertigo and sleepiness were the most common effects in both groups.

Therefore, the combination of rizatriptan and trimebutine is conspicuously more effective and reveals to be faster in the time for relieving migraine when compared to the treatment with rizatriptan alone. This combination does not increase the adverse events nor does it increase the recurrence of migraine.

An example of the preferred embodiment has been described, but it should be understood that the scope of the present invention encompasses other possible variations, being limited only by the content of the appended claims, including therein the possible equivalents.

1. A pharmaceutical combination, characterized by comprising a therapeutically effective amount of a triptan, a non-steroidal anti-inflammatory drug or simple analgesic, and trimebutine for the treatment of attacks of headache and migraine.

2. A pharmaceutical combination according to claim 1, characterized in that said compounds are arranged individually into three different tablets or capsules, which are administered simultaneously and randomly.

3. A pharmaceutical combination according to claim 1, characterized in that said compounds are arranged in a single tablet or capsule.

4. A pharmaceutical combination according to claim 1, characterized in that:
(i) the triptan is selected from the group consisting of sumatriptan, naratriptan, zolmitriptan, rizatriptan, eletriptan, almotriptan and frovatriptan and mixtures thereof;
(ii) the non-steroidal anti-inflammatory drug is selected from the group consisting of meloxican, ibuprofen, lysine clonixinate, indometacin, naproxen sodium, rofecoxib, celecoxib and cetoprofen;
(iii) the simple analgesic is selected from the group consisting of paracetamol and acetylsalicylic acid and salicylates; and
(iv) the trimebutine is the hydrogenated maleate of 2-dimethyl-amino-2-phenylbutyl-3,4,5-trimetoxybenzoate or its stereoisomers.

5. A pharmaceutical combination according to claim 4, characterized in that the triptans are selected from sumatriptan, naratriptan and rizatriptan.

6. A pharmaceutical combination according to claim 4, characterized in that sumatriptan is present in the range of 40 to 60 mg, naratriptan is present in the range of 3 to 4 mg and rizatriptan is present in the range of 8 to 12 mg.

7. A pharmaceutical combination according to claim 6, characterized in that sumatriptan is present in the range of 45 to 55 mg, naratriptan is present in the range of 3.5 to 4 mg e rizatriptan is present in the range of 9 to 11 mg.

8. A pharmaceutical combination according to claim 5, characterized in that the triptan is sumatriptan.

9. The pharmaceutical combination according to claim 4, characterized in that meloxican is present in the range of 8 to 20 mg, ibuprofen is present in the range of 500 to 1000 mg, lysine clonixinate is present in the range of 200 to 300 mg, indometacin is present in the range of 50 to 80 mg, naproxen sodium is present in the range of 500 to 1000 mg, rofecoxib is present in the range of 40 to 60 mg, celecoxib is present in the range of 180 to 260 mg and cetoprofen is present in the range of 50 to 120 mg.

10. A pharmaceutical combination according to claim 9, characterized in that meloxican is present in the range of 10 to 20 mg, ibuprofen is present in the range of 600 to 800 mg, lysine clonixinate is present in the range of 250 to 300 mg, indometacin is present in the range of 60 to 70 mg, naproxen sodium is present in the range of 500 to 600 mg, rofecoxib is present in the range of 45 to 55 mg, celecoxib is present in the range of 200 to 250 mg and cetoprofen is present in the range of 50 to 100 mg.

11. A pharmaceutical combination according to claim 9, characterized in that the non-steroidal anti-inflammatory drug is meloxican.

12. A pharmaceutical combination according to claim 4, characterized in that paracetamol, acetylsalicylic acid and salicylates are present in the range of 500 to 1000 mg.

13. A pharmaceutical combination according to claim 12, characterized in that paracetamol is present in the range of 700 to 1000 mg.

14. A pharmaceutical combination according to claim 12, characterized in that acetylsalicylic acid and salicylates are present in the range of 700 to 900 mg.

15. A pharmaceutical combination according to claim 4, characterized in that the trimebutine is present in the range of 150 to 250 mg.

16. A pharmaceutical combination according to claim 15, characterized in that the trimebutine is present in the range of 180 to 220 mg.

17. A pharmaceutical combination according to claim 1, characterized in that the headache is selected from the group consisting of migraine, tension-type headache, cluster headache, new daily and persistent headache, hemicrania continua, chronic paroxysmal hemicrania, acute high-altitude headaches, headaches from alcohol ingestion, from simple general pathologies such as flues and common colds.

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)
